(19) United States
(12) Patent Application Publication
(10) Pub. No.: US 2024/0309099 A1
(43) Pub. Date: Sep. 19, 2024

(54) BISPECIFIC TETRAVALENT ANTIBODY TARGETING EGFR AND HER3

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Dennis R. GOULET, Redmond, WA (US); Jahan KHALILI, Kirkland, WA (US); Blair Renshaw, Renton, WA (US); Nga Sze Amanda MAK, Mukilteo, WA (US); Hai ZHU, Bothell, WA (US); Yi ZHU, Chengdu, Sichuan (CN)

(21) Appl. No.: 18/681,827

(22) PCT Filed: Aug. 25, 2022
(86) PCT No.: PCT/US2022/075445

§ 371 Date: Feb. 6, 2024

Related U.S. Application Data
(60) Provisional application No. 63/237,033, filed on Aug. 25, 2021.

(51) Int. Cl. C07K 16/28; A61K 45/06; A61K 47/68; A61P 35/00; C07K 16/32

(52) U.S. Cl. CPC C07K 16/2863; A61K 45/06; A61K 47/6849; A61P 35/00; C07K 16/32; C07K 2317/31; C07K 2317/55; C07K 2317/622; C07K 2317/73

(57) ABSTRACT

A bispecific antibody comprises two sets of heavy and light chains, wherein each set of the heavy chain and the light chain form a Fab region having a binding specificity to EGFR; the antibody and further comprises a scFv domain covalently linked to N-terminal of the heavy chain, N-terminal of the light chain, or C-terminal of the light chain, wherein the scFv domain has a binding specificity to HER3.

### CROSS REFERENCE TO RELATED APPLICATIONS

This application claims the benefit of the filing date of U.S. Provisional Application Ser. No. 63/237,033 filed Aug. 25, 2021, under 35 U.S.C. 119(e), the entire disclosures of which are incorporated by reference herein.

### TECHNICAL FIELD

The present disclosure generally relates to the technical field of antibody cancer therapeutics, and more particularly relates to bispecific tetravalent antibodies.

### BACKGROUND

The human epidermal growth factor receptor (EGFR, also known as ErbB1, HER1) family has four members, EGFR, HER2, HER3, and HER4. Deregulation of each member of the family by means of mutation, amplification, and overexpression plays an important role in tumorigenesis and tumor metastasis. Overexpression is associated with the development of a wide variety of tumors, including but not limited to breast, ovarian, stomach, and gastric cancer, adenocarcinoma of lung, aggressive forms of uterine cancer, and salivary duct carcinomas. In the case of breast cancer, the overexpression of HER2 occurs in 30% of breast cancer patients, and the underlying HER2 mutation and amplification produce aberrant growth signals that activate its downstream signaling pathway leading to tumorigenesis. Of the subtypes of breast cancer tested negative for HER2, EGFR is overexpressed in at least 50%of triple negative breast cancer (test negative for estrogen and progesterone receptors and HER2 protein). HER3 is overexpression in approximately 20-30% of invasive forms of breast cancer. HER3 is the only member in the family that is catalytically inactive and requires dimerization with other members to be activated. For example, HER3 may dimerizes with HER2 on the surface of tumor cells, which activates PI3K/AKT signalling that promotes tumor growth and survival.

Interruption of EGFR signaling, either by blocking EGFR binding sites on the extracellular domain of the receptor or by inhibiting intracellular tyrosine kinase activity, can prevent the growth of EGFR-expressing tumors and improve the patient's condition. Several anti-EGFR antibodies, including cetuximab, panitumumab and nimotuzumab, are approved for treating metastatic colorectal cancer, head and neck squamous cell carcinoma, and glioma (Price and Cohen, 2012; Bode et al. 2012). Trastuzumab (Herceptin) and other agents targeting HER2 have antitumor efficacy in patients with HER2-expressing breast cancer and stomach cancer. However, Trastuzumab is effective only in cancers where HER2 is overexpressed. Many tumors that initially respond to these therapeutic agents eventually progress due to an acquired resistance to the agents, and the long-term benefit seems to be limited in some patients. In the case of HER2-targeted therapies, the resistance can occur via upregulation of HER3 or its ligand HRG. And yet, the current therapeutic approaches aiming at inhibiting the activation of HER2/HER3 signalling pathway have failed to provide meaningful clinical benefit (Geuijen et al. 2018; Yu et al. 2019). The present disclosure is related to methods of making and using bispecific tetravalent antibodies targeting EGFR and HER3 for treating patients with cancer.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The disclosure provides bispecific tetravalent antibodies targeting two members of EGFR family, EGFR and HER3, and the methods for making and using the antibodies. The bispecific tetravalent antibodies may include an immunoglobulin G (lgG) moiety with two heavy chains and two light chains, and two scFv moieties being covalently connected to N terminal of the heavy chain, or either N or C terminal of the light chain. The IgG moiety may have a binding specificity to a first member of EGFR family. The scFv moiety may have a binding specificity to a second member of the EGFR family. The IgG moiety and two scFv moieties are covalently connected to be functional as a bispecific antibody. The objectives and advantages of the disclosure will become apparent from the following detailed description of preferred embodiments thereof in connection with the accompanying drawings.

In one aspect, the application provides a bispecific antibody, comprising two sets of heavy and light chains. Each set of the heavy chain and the light chain form a Fab region having a binding specificity to EGFR. The antibody may further comprise a scFv domain covalently linked to N-terminal of the heavy chain, N-terminal of the light chain, or C-terminal of the light chain. The scFv domain has a binding specificity to HER3. In one embodiment, the bispecific antibody comprises an IgG domain. In one embodiment, the bispecific antibody comprises an IgG1 domain.

In one embodiment, the scFv domain may be linked to the N-terminal of the heavy chain. In one embodiment, the scFv domain may be linked to the N-terminal or C-terminal of the light chain.

In one embodiment, the scFv domain is linked to the N-terminal or C-terminal of the light chain, wherein the light chain comprises an amino acid sequence having a sequence identity to SEQ ID NO. 1, 3, 5, 7, or 9.

In one embodiment, the antibody may include an amino acid sequence having at least 50%, 60%, 70%, 75%, 80%, 85%, 90%, 95%, 98%, or 99%of sequence identity to SEQ ID NO 17, 23, or 24.

In one embodiment, the scFv domain is linked to the N-terminal of the heavy chain, wherein the heavy chain comprises an amino acid sequence having a sequence identity to SEQ ID NO. 2, 4, 6, 8, or 10.

In one embodiment, the antibody may include an amino acid sequence having at least 50%, 60%, 70%, 75%, 80%, 85%, 90%, 95%, 98%, or 99%of a sequence identity to SEQ ID NO. 22.

In one embodiment, the heavy chain may include 3 complementary determining regions (CDRs) having the amino acid sequence of SEQ ID NO 31, 32, and 33. In one embodiment, the heavy chain may include 3 CDRs having the amino acid sequence of SEQ ID NO 37, 38, and 39.

In one embodiment, the light chain may include 3 CDRs having the amino acid sequence of SEQ ID NO 34, 35, and 36. In one embodiment, the light chain may include 3 CDRs having the amino acid sequence of SEQ ID NO 40, 41, and 42.

In one embodiment, the antibody may include an IgG constant region, wherein the IgG constant region comprises an amino acid sequence having at least 50%, 60%, 70%, 75%, 80%, 85%, 90%, 95%, 98%, or 99%of sequence identity to SEQ ID NO. 19.

In one embodiment, the antibody may include a kappa constant region, wherein the kappa constant region comprises an amino acid sequence having at least 50%, 60%, 70%, 75%, 80%, 85%, 90%, 95%, 98%, or 99% of sequence identity to SEQ ID NO. 20.

In one embodiment, the scFv domain may include an amino acid sequence having at least 50%, 60%, 70%, 75%, 80%, 85%, 90%, 95%, 98%, or 99%of sequence identity to SEQ ID NO. 11, 12, 13, 14, 15, or 16.

In one embodiment, the scFv domain may include a variable light chain (VL), wherein the VL has an amino acid sequence having at least 50%, 60%, 70%, 75%, 80%, 85%, 90%, 95%, 98%, or 99% of sequence identity to SEQ ID NO. 11, 13, or 15. In one embodiment, the VL comprises CDRs having an amino acid SEQ ID NO. 46, 47, and 48.

In one embodiment, the scFv domain may include a variable heavy chain (VH), wherein the VH has an amino acid sequence having at least 50%, 60%, 70%, 75%, 80%, 85%, 90%, 95%, 98%, or 99%of sequence identity to SEQ ID NO. 12, 14, or 16. In one embodiment, the V_{H} comprises CDRs having an amino acid SEQ ID NO. 43, 44, and 45.

In one embodiment, the scFv domain may have a configuration of VLVH or VHVL from the N terminal to the C terminal. In one embodiment, the scFv may include a disulphide bond between V_{L} and V_{H}. In one embodiment, the disulfide bond may be between vL 100 and vH44 (Kabat) of the scFv domain. In one embodiment, the scFv may include R19S (Kabat) mutation.

In one embodiment, the scFv domain comprise V_{L} having an amino acid sequence having a sequence identity to SEQ ID NO. 11 and V_{H} having an amino acid sequence having sequence identity to SEQ ID NO. 12. In one embodiment, the scFv comprise V_{L} having an amino acid sequence having a sequence identity to SEQ ID NO. 13 and V_{H} having an amino acid sequence having sequence identity to SEQ ID NO. 14. In another embodiment, the scFv comprise V_{L} having an amino acid sequence having a sequence identity to SEQ ID NO. 15 and V_{H} having an amino acid sequence having sequence identity to SEQ ID NO. 16.

In one embodiment, the antibody may include an amino acid sequence having at least 50%, 60%, 70%, 75%, 80%, 85%, 90%, 95%, 98%, or 99% of sequence identity to SEQ ID NO. 18, and the antibody may include an amino acid sequence having at least 50%, 60%, 70%, 75%, 80%, 85%, 90%, 95%, 98%, or 99% of sequence identity to SEQ ID NO. 17.

In one embodiment, the antibody may include an amino acid sequence having at least 50%, 60%, 70%, 75%, 80%, 85%, 90%, 95%, 98%, or 99% of sequence identity to SEQ ID NO. 22, and the antibody may include an amino acid sequence having at least 50%, 60%, 70%, 75%, 80%, 85%, 90%, 95%, 98%, or 99% of sequence identity to SEQ ID NO. 21.

In one embodiment, the antibody may include an amino acid sequence having at least 50%, 60%, 70%, 75%, 80%, 85%, 90%, 95%, 98%, or 99% of sequence identity to SEQ ID NO. 18, and the antibody may include an amino acid sequence having at least 50%, 60%, 70%, 75%, 80%, 85%, 90%, 95%, 98%, or 99% of sequence identity to SEQ ID NO. 23.

In one embodiment, the antibody may include an amino acid sequence having at least 50%, 60%, 70%, 75%, 80%, 85%, 90%, 95%, 98%, or 99%o f sequence identity to SEQ ID NO. 25, and the antibody may include an amino acid sequence having at least 50%, 60%, 70%, 75%, 80%, 85%, 90%, 95%, 98%, or 99% of sequence identity to SEQ ID NO. 24.

In another aspect, the application provides an isolated nucleic acid encoding the bispecific antibody as disclosed herein.

In a further aspect, the application provides an expression vector including the isolated nucleic acid encoding the bispecific antibody as disclosed herein. In one embodiment, the expression vector may be expressible in a cell.

In a further aspect, the application provides a host cell comprising the nucleic acid as disclosed herein.

In a further aspect, the application provides methods of producing the bispecific antibody as disclosed herein. The method includes the step of culturing the host cell as disclosed herein so that the bispecific antibody is produced.

In a further aspect, the application provides immunoconjugates comprising the bispecific antibody and a cytotoxic agent, and wherein the cytotoxic agent comprises a chemotherapeutic agent, a growth inhibitory agent, a toxin, or a radioactive isotope.

In a further aspect, the application provides pharmaceutical compositions, comprising the bispecific antibody and a pharmaceutically acceptable carrier. In one embodiment, the pharmaceutical composition may include radioisotope, radionuclide, a toxin, a therapeutic agent, a chemotherapeutic agent or a combination thereof. In one embodiment, the pharmaceutical composition may include the immunoconjugate and a pharmaceutically acceptable carrier.

In a further aspect, the application provides methods of treating a subject with a cancer. In one embodiment, the method may include the step of administering to the subject an effective amount of the bispecific antibody. In one embodiment, the cancer may include cells expressing EGFR, HER3 or both. In one embodiment, the cancer may include breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukaemia, myeloma.

In one embodiment, the method further includes co-administering an effective amount of a therapeutic agent.

In one embodiment, the therapeutic agent may include an antibody, a chemotherapy agent, an enzyme, or a combination thereof. In one embodiment, the therapeutic agent may include capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

In one embodiment, the subject is a human.

In a further aspect, the application provides a solution comprising an effective concentration of the bispecific antibody. In one embodiment, the solution is blood plasma in a subject.

### BRIEF DESCRIPTION OF THE DRAWINGS

The foregoing and other features of this disclosure may become more fully apparent from the following description and appended claims, taken in conjunction with the accompanying drawings. Understanding that these drawings depict only several embodiments arranged in accordance with the disclosure and are, therefore, not to be considered limiting of its scope, the disclosure may be described with additional specificity and detail through use of the accompanying drawings, in which:

FIG. 1 depicts the configuration of bispecific tetravalent antibodies targeting EGFR and HER3, i.e., EGFR×HER3 bispecific antibodies;

FIG. 2 depicts thermal stability data for EGFR×HER3 bispecific antibodies as measured by dynamic light scattering;

FIG. 3 shows biolayer interferometry sensorgrams for EGFR×HER3 bispecific antibodies binding to human EGFR;

FIG. 4 shows biolayer interferometry sensorgrams for EGFR×HER3 bispecific antibodies binding to human HER3; and

FIG. 5 depicts the effect of EGFR x HER3 bispecific antibodies on proliferation of FaDu tumor cells.

### DETAILED DESCRIPTION

This disclosure provides bispecific tetravalent antibodies with superior therapeutic properties or efficacies over the currently known anti-EGFR antibodies. In one embodiment, the antibodies target members of EGFR family including, without limitation, EGFR and HER3. These bispecific tetravalent antibodies may inhibit different receptor-mediated oncogenic signaling simultaneously therefore overcome resistance in EGFR inhibitor or monoclonal antibody treatment.

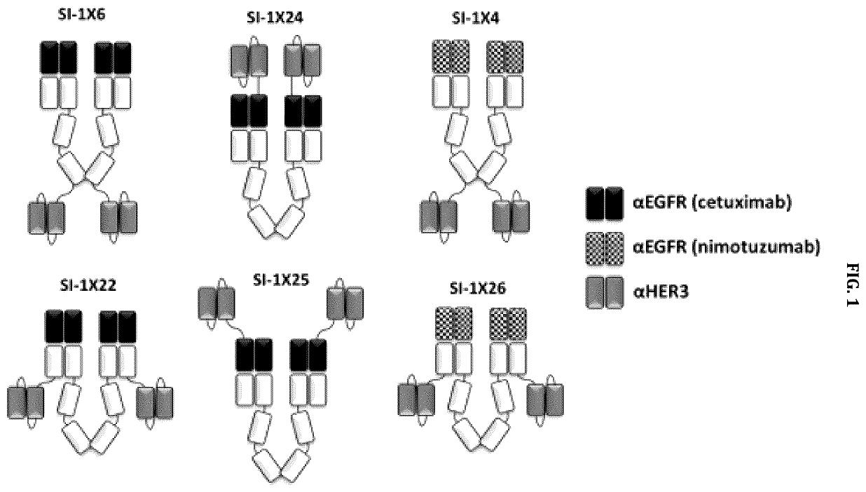

The present disclosure provides, among others, isolated antibodies or antigen binding fragments, humanized antibodies or antigen binding fragments, methods of making such antibodies or antigen binding fragments, monoclonal and/or recombinant monospecific antibodies, multi-specific antibodies, antibody-drug conjugates and/or immuno-conjugates composed from such antibodies or antigen binding fragments, pharmaceutical compositions containing the antibodies, monoclonal and/or recombinant monospecific antibodies, multi-specific antibodies, antibody-drug conjugates and/or immuno-conjugates, the methods for making the antibodies and compositions, and the methods for treating cancer using the antibodies and compositions disclosed herein. Specifically, the present disclosure provides a group of bispecific tetravalent antibodies with their binding specificity to human EGFR and HER3, also known as EGFR x HER3 bispecific antibodies (FIG. 1), wherein an isolated antibody comprises an amino acid sequence having an identity with a sequence selected from SEQ ID NO. 17, 22, 23, 24.

The term “antibody” is used in the broadest sense and specifically covers single monoclonal antibodies and/or recombinant antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, multi-specific or multi-effective, human and humanized antibodies, as well as active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above.

The term “Fv” refers to the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) can recognize and bind antigen, although at a lower affinity than the entire binding site.

In some embodiments, antibody may include immunoglobulin molecules and immunologically active portions of immunoglobulin molecules, i.e., molecules that contain a binding site and that immunospecifically bind an antigen. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The light chains of antibodies (immunoglobulins) from any vertebrate species can be assigned to one of two clearly distinct types, called kappa and lambda, based on the amino acid sequences of their constant domains. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any method. For example, the monoclonal antibodies to be used in accordance with the present disclosure may be made by the hybridoma method first described by Kohler & Milstein, Nature, 256:495 (1975), or may be made by recombinant DNA methods (e.g., U.S. Pat. No. 4,816,567). “Recombinant” means the antibodies are generated using recombinant nucleic acid techniques in exogeneous host cells.

Monoclonal antibodies can be produced using various methods, including without limitation, mouse hybridoma, phage display, recombinant DNA, molecular cloning of antibodies directly from primary B cells, and antibody discovery methods (see Siegel. Transfus. Clin. Biol. 2002; Tiller. New Biotechnol. 2011; Seeber et al. PLOS One. 2014). Monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

The term “multi-specific, multivalent” antibody as used herein denotes an antibody that has at least two binding sites each having a binding affinity to an epitope of an antigen. The term “bispecific, tetravalent antibody” as used herein denotes an antibody that has four antigen-binding sites specific to two antigens. For example, the antibodies disclosed herein are bispecific tetravalent to EGFR and HER3.

The term “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. [see, e.g., Queen et al., Proc. Natl Acad Sci USA, 86:10029-10032 (1989), Hodgson et al., Bio/Technology, 9:421 (1991)).

The terms “antigen-or epitope-binding portion or fragment”, “variable domain”, “variable region”, “variable region sequence”, or “binding domain” refer to fragments of an antibody that are capable of binding to an antigen (such as EGFR and HER3 in this application). The antigen-binding fragment (Fab) is a region (Fab region) on an antibody that binds to antigens. These fragments may be capable of the antigen-binding function and additional functions of the intact antibody. Examples of binding fragments include, but are not limited to, a single-chain Fv fragment (scFv) consisting of the variable light chain (VL) and variable heavy chain (VH) domains of a single arm of an antibody connected in a single polypeptide chain by a synthetic linker, or a Fab fragment which is a monovalent fragment consisting of the VL, constant light (CL), VH and constant heavy 1 (CH1) domain.

Antibody fragments can be even smaller sub-fragments and can consist of domains as small as a single CDR domain, the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies.

The “antigen-or epitope-binding portion or fragment”, “variable region”, “variable region sequence”, or “binding domain” may be derived from an antibody of the present application by several art-known techniques. For example, purified monoclonal antibodies can be cleaved with an enzyme, such as pepsin, and subjected to HPLC gel filtration. Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab')2 fragment that has two antigen combining sites and is still capable of cross-linking antigen. The appropriate fraction containing Fab fragments can then be collected and concentrated by membrane filtration and the like. For further description of general techniques for the isolation of active fragments of antibodies (see for example, Khaw, B. A. et al. J. Nucl. Med. 23:1011-1019 (1982); Rousseaux et al. Methods Enzymology, 121:663-69, Academic Press, 1986).

The terms “isolated” or “purified” refers to a biological molecule free from at least some of the components with which it naturally occurs. Either “Isolated” or “purified,” when used to describe the various polypeptides disclosed herein, means a polypeptide that has been identified and separated and/or recovered from a cell or cell culture from which it was expressed. Ordinarily, a purified polypeptide will be prepared by at least one purification step. An “isolated” or a “purified” antibody refers to an antibody which is substantially free of other antibodies having different antigenic a binding specificity.

The terms “a”, “an” and “the” as used herein are defined to mean “one or more” and include the plural unless the context is inappropriate.

The terms “polypeptide”, “peptide”, and “protein”, as used herein, are interchangeable and are defined to mean a biomolecule composed of amino acids linked by a peptide bond.

The term “antigen” refers to an entity or fragment thereof which can induce an immune response in an organism, particularly an animal, more particularly a mammal including a human. The term includes immunogens and regions thereof responsible for antigenicity or antigenic determinants.

The term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated. While the immunogenic response generally includes both cellular (T cell) and humoral (antibody) arms of the immune response, antibodies directed against therapeutic proteins (anti-drug antibodies, ADA) may consist of IgM, IgG, IgE, and/or IgA isotypes.

The terms “specific binding”, “specifically binds to”, or “is specific for a particular antigen or an epitope” means that the binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “affinity” refers to a measure of the attraction between two polypeptides, such as antibody/antigen, receptor/ligand, etc. The intrinsic attraction between two polypeptides can be expressed as the binding affinity equilibrium dissociation constant (KD) of a particular interaction. A KD binding affinity constant can be measured, e.g., by Bio-Layer Interferometry, where KD is the ratio of kdis (the dissociation rate constant) to kon (the association rate constant), as KD=kdis/kon.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to the equilibrium dissociation constant of a particular antibody-antigen interaction. Typically, an antibody that specifically binds an antigen will have a KD that is 20-, 50-, 100-, 500-, 1000-, 5,000-, 10,000-or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1: Configuration of EGFR×HER3 bispecific antibodies

The cancer-associated gain-of-function mutations alter the HER3 kinase domain and ultimately enhance allosteric function, which provides a structural and mechanistic basis for developing drugs that target EGFR/HER3 dimerization. Inhibiting EGFR/HER3 signaling may be achieved by using either small molecule drugs or monoclonal antibodies against members of the EGFR family. For example, both cetuximab and nimotuzumab are anti-EGFR antibodies that have been proved to be therapeutically effective in clinical trials. The binding fragment derived from these antibodies may be referred as a therapeutic binding domain because their anti-tumor proliferation activity is proven in clinical trials. Despite the progress in combination therapy involving the use of two therapeutic antibodies, there is need to develop a single efficacious bispecific antibody for inhibiting EGFR/HER3 dimerization. Of concerns, the precise geometry of the bispecific antibody (i.e., spacing, and relative configuration of the two sets of binding domains) may significantly impact the properties of the therapeutic agent in terms of, for example, expression titer, stability, antigen binding, or efficacy at inhibiting proliferation or impacting another biological function.

FIG. 1 depicts configurations of six bispecific tetravalent antibodies, each of these EGFR×HER3 bispecific antibodies comprise an immunoglobulin G (lgG) moiety with two heavy chains and two light chains and two scFv binding domains being covalently connected to two designated ends of the antibody via a linker, such as (Gly-Gly-Gly-Gly-Ser)n linkers or a (Gly-Gly-Gly-Ser)n linkers, or (GmS)n linkers. Of this panel of EGFR×HER3 bispecific antibodies, SI-1X6 and SI-1X4 have been characterized by having an anti-EGFR Fab region and an anti-HER3 scFv domain linked d to the C-terminus of heavy chain (HC)(WO2016106157A1, incorporated herein by reference in its entirety). The two antibodies share the same configuration that keeps the two binding domains apart at the two ends of HC with at least CH1, CH2, and CH3 in between. In comparison, SI-1X22, SI-1X24, SI-1X25, and SI-1X26 are configured to have the anti-HER3 scFv domain linked to either one end of LC or N-terminus of HC. As a result, the space between two binding domains is reduced to either a CH1 when the scFv domain is linked to C-terminus of LC (SI-1X22 and SI-1X26) or none when the scFv domain is linked to N-terminus of either HC or LC (SI-1X24 and SI-1x25).

While each domain may exert independent binding specificity, keeping the two binding domains physically closer may improve the efficiency of antibody binding to both EGFR and HER3 on the same tumor cell. For example, the proximity of the binding domains may instill a more rigid conformation where steric constraints prevent domains from rearranging in such a way as to allow for dimerization of EGFR and HER3. In contrast, a long inter-domain physical distance combined with flexible regions between binding domains, may allow for the conformational flexibility to cause undesired receptor dimerization and downstream proliferative signaling. The mutation R19S (Kabat) in the VH of scFvs on the light chain (WO2021092266A1, incorporated herein by reference in its entirety) was used to prevent binding of light chain components to protein A during purification. When the anti-HER3 scFv domain was fused to a light chain, the paired VH/VL within the Fab were stabilized with a disulfide staple (VH 44C/VL 100C, Kabat).

### Example 2: Generation of EGFRxHER3 bispecific antibodies

SI-1X22, SI-1X24, SI-1X25, and SI-1X26 were cloned and purified. Genes encoding antibody heavy and light chains (preceded by Kozak and secretory signal peptide) were cloned into pTT5 vector using standard molecular biology techniques. Antibodies and were expressed by transiently transfecting the expression plasmids for heavy and light chains in the ExpiCHO system (Thermo Fisher). Briefly, 10 μg of each expression plasmid was brought to 1ml with OptiPRO SFM medium. 1ml of OptiPRO SFM medium containing 80 ul Expifectamine CHO reagent was added to the DNA and incubated at room temperature for 2.5 minutes. The resulting mixture was then added to 25ml ExpiCHO cells at 6x106 cells/ml in a 125ml Erlenmeyer flask and incubated at 37° C., 5% CO₂, 150 rpm. Cells were fed with 8.75 ml ExpiCHO feed and 150 μl of CHO enhancer at 24 hours post-transfection and shifted to 32° C., 5% CO₂, 150rpm. Cells were fed again at 48 hours post-transfection with 8.75 ml ExpiCHO feed. Culture supernatant was harvested 9 days post-transfection, spun for 1 hour at 4500 rpm to pellet the cells and then passed through a 0.2 mm filter. Expression titer was quantitated using biolayer interferometry on an Octet384 system with protein A sensors and a standard curve prepared with purified bispecific antibody protein.

Proteins were purified from the harvested supernatant using a 1-ml MabSelect PrismA protein A column (GE Healthcare). The column was equilibrated with phosphate-buffered saline. The supernatant was then passed through the column at a flow rate of 1 ml/min. The column was washed with 10ml PBS. Protein was then eluted by passing 5 ml of 50 mM sodium acetate, pH 3.5 through the column. The eluted protein was immediately neutralized by addition of 0.5ml 1M Tris-Cl, pH8.0.

Immediately after first-step protein A or His tag purification, proteins were analyzed by analytical SEC using Waters Acquity UPLC H-Class with ACQUITY UPLC® Protein BEH SEC 200Å, 4.6 mm×150 mm, 1.7 μm column. PBS (125 mM sodium phosphate, 137 mM sodium chloride, pH 6.8) was used as mobile phase for 10-minute runs at 0.3 ml/min, injecting 10 μg protein. Proteins were further purified by preparative SEC using Superdex Increase 10/300 GL column in mobile phase of 25 mM sodium acetate, 125 mM NaCl, pH 5.5, ultimately to be buffer-exchanged into 25 mM sodium acetate, 125 mM NaCl, 10% sucrose, pH 5.5. Final samples contained >95% protein of interest (POI) as assessed by analytical SEC and were used for subsequent assays.

### Example 3: Protein stability

Protein stability is a key parameter defined by the difference in free energy between the folded and unfolded states. For protein therapeutics, stability may impact immunogenicity, pharmacokinetics, and even efficacy, and reduction of aggregation can help to develop therapeutics that are easier to manufacture and safer for patients. In addition, expression efficiency and protein yield directly determine the cost of protein therapeutics. If proteins can be more efficiently expressed to reach higher titers and increased yield of purified protein, manufacturing costs can be reduced significantly.

After transient expression in ExpiCHO cells, titer of the bispecific antibodies was quantitated using biolayer interferometry. As shown in Table 1, the data demonstrate that all proteins expressed in the ExpiCHO expression system, indicating they are stable enough to be efficiently produced. For antibodies with nimotuzumab variable regions (SI-1X4 and SI-1X26), titer was comparable. For antibodies with cetuximab variable regions (SI-1X6, SI-1X22, SI-1X24, and SI-1X25), titer was higher than for nimotuzumab-based antibodies, and was highest for SI-1X24 which contains anti-HER3 scFv at the N-terminus of the cetuximab heavy chain.

Another parameter related to protein stability is the amount of aggregation after first step affinity purification. Antibodies with higher stability tend to have lower aggregation, and therefore higher % POI (percentage protein of interest) by analytical size-exclusion chromatography. After protein A purification, the bispecific antibodies were analyzed by analytical SEC to check for aggregation (see Table 1). Of the antibodies containing nimotuzumab variable regions (SI-1X4 and SI-1X26), SI-1X4 containing anti-HER3 scFv at the C-terminus of the nimotuzumab heavy chain had significantly less aggregation (and therefore higher % POI). For cetuximab-based antibodies (SI-1X6, SI-1X22, SI-1X24, and SI-1X25), aggregation was lowest for SI-1X24 (containing anti-HER3 scFv at the N-terminus of the heavy chain) which had the highest % POI after purification.

### Example 4: Thermal stability

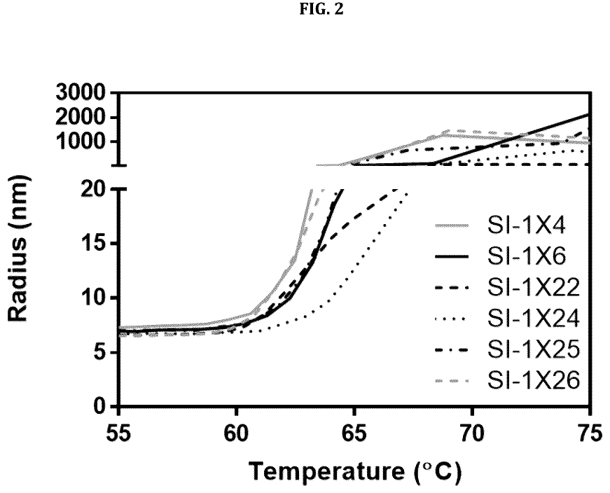

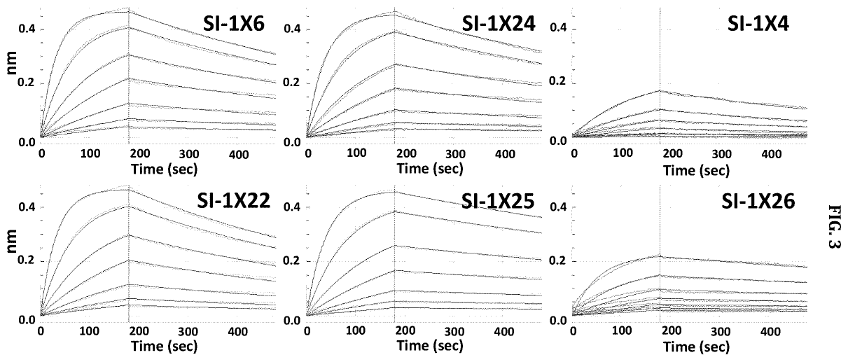

Thermal stability is another parameter for assessing the quality of any antibody. Dynamic light scattering was used to compare the thermal stability of the EGFRxHER3 bispecific antibodies. In thermal stability experiments, the temperature was ramped from 25° C. to 85° C. at 0.5° C./min while the radius of the proteins (1 mg/ml) was monitored by a Wyatt DynaPro Plate Reader III. As shown in FIG. 2, the particle size increase is indicative of protein aggregation or other unfolding events. As an objective measure of thermal stability, the temperature at which the radius surpassed 10 nm was tabulated (Table 1). Of the cetuximab-based antibodies (SI-1X6, SI-1X22, SI-1X24, SI-1X25), SI-1X24 was the most stable in the assay with a Tm of 64.75° C. The other three antibodies in the family (SI-1X6, SI-1X22, SI-1X25) had similar Tms in the range of 62-63° C. Thus, for cetuximab-based bispecific antibodies, the position of the anti-HER3 scFv can cause significant differences in thermal stability. As for the nimotuzumab-based antibodies (SI-1X4, SI-1X26), both antibodies unfolded at about 61.5° C., indicating that these two molecules have similar thermal stability.

### Example 5: Octet binding

Sartorius Octet platform applies Bio-Layer Interferometry (BLI) as a label-free technology for measuring protein-protein interactions. It is an optical analytical technique that analyses the interference pattern of white light reflected from two surfaces: a layer of immobilized protein on the biosensor tip, and an internal reference layer. Any change in the number of molecules bound to the biosensor tip causes a shift in the interference pattern that can be measured in real-time. In this method, the binding between an antibody/Fc containing protein immobilized on the Anti-human IgG Fc Capture (AHC) Biosensors tip surface and an antigen in solution produces an increase in optical thickness at the biosensor tip, which results in a wavelength shift, Δλ, directly reflecting the change in thickness of the biological layer. The interaction of these two molecules is measured in real time, providing the ability to monitor binding specificity, rates of association and dissociation, or concentration, with precision and accuracy. Unbound molecules, changes in the refractive index of the surrounding medium, or changes in flow rate do not affect the interference pattern.

Biolayer interferometry (Octet) binding assays were performed on an Octet384 instrument to quantify binding kinetics of bispecific antibodies to EGFR and HER3. Antibody was captured to anti-human Fc (AHC) sensor tips by loading for 180 seconds at 5 μg/ml. After a 60-second baseline step, a 180-second association phase with serial dilutions (0-100 nM; 1:2 dilution factor) of His-tagged EGFR (expressed/purified in-house) or HER3 (purchased from Acro Bio) in assay buffer (phosphate-buffered saline containing 0.1% BSA, 0.05% Tween20) was performed, followed by a 300-second dissociation phase in assay buffer. Regeneration was achieved using 10 mM glycine, pH 1.5. Binding curves were globally fit to a 1:1 model to extract the dissociation constants, K_{D}, and kinetic association and dissociation rates.

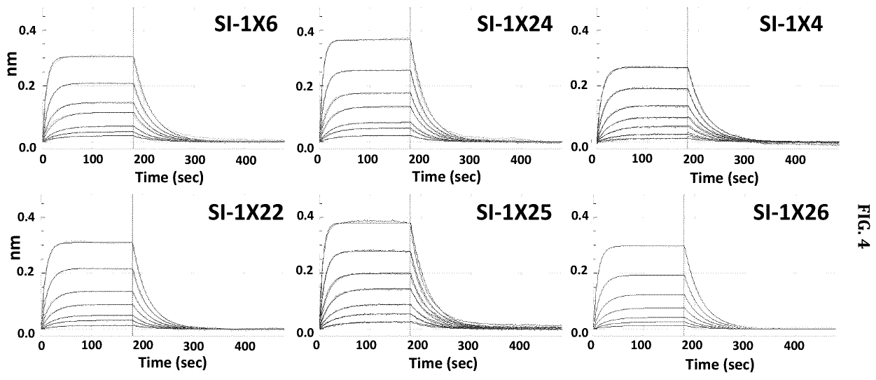

Biolayer interferometry was used to measure binding kinetics for EGFRxHER3 bispecific antibodies to human EGFR. As shown in FIG. 3 and Table 2, the EGFR binding data reveal that all the cetuximab-based antibodies (SI-1X6, SI-1X22, SI-1X24, SI-1X25) had similar KD values ranging from 3 to 6 nM, while nimotuzumab-based antibodies (SI-1X4, SI-1X26) had weaker affinity with KD values of 11 to 24 nM. Cetuximab-based antibodies had higher binding response in the assay, which is also suggestive of stronger binding. The difference in EGFR binding within the two families (cetuximab and nimotuzumab) was not significant.

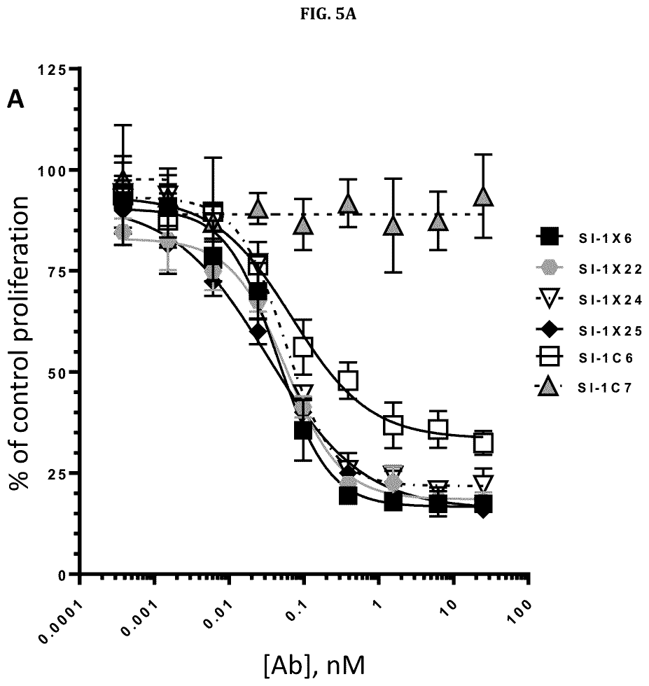

Biolayer interferometry was used to measure binding kinetics for EGFR x HER3 bispecific antibodies to human HER3. As shown in FIG. 4 and Table 3, the HER3 binding data reveal that all the bispecific antibodies (SI-1X4, SI-1X6, SI-1X22, SI-1X24, SI-1X25, SI-1X26) had similar KD values ranging from 94 to 164 nM. This similarity of HER3 binding makes sense since the HER3-binding domain is derived from the same antibody for all the proteins. The result suggests that the anti-HER3 scFv can be placed into any position of cetuximab-and nimotuzumab-based bispecific antibodies without significant differences in in vitro binding.

### Example 6: Inhibiting tumour cell proliferation

To evaluate the effect of EGFR×HER3 bispecific antibodies on cell growth, a proliferation assay with FaDu cells was conducted with Alamar Blue used to quantify proliferation. The hypopharyngeal squamous cell carcinoma line FaDu was purchased from ATCC (cat #HTB-43) and was maintained in EMEM medium supplemented with 10% fetal bovine serum at 37° C. with 5% CO₂. FaDu cells were detached from flasks with trypsin and diluted to 1.2×10⁵ cells/ml in EMEM medium+1% FBS. 50 ml of cell suspension (6000 cells) was seeded to interior 60 wells of 96-well tissue culture plates. Outer wells were filled with 300ml sterile H20 to minimize evaporation in interior wells. Cells were allowed to adhere for 4 hours at 37° C., 5% CO₂. Antibodies to be tested were diluted to 2X final concentration in EMEM medium+1% FBS. 50ml of test antibodies were added to each well for a total volume of 100 ml per well. Each antibody was tested in triplicate at the following final concentrations: 25 nM, 6.25 nM, 1.563 nM, 0.391 nM, 0.098 nM, 0.024 nM, 0.006 nM, 0.0015 nM, and 0.0004 nM. Each plate contained two antibodies at those concentrations tested in triplicate. Six control wells per plate contained cells with medium only. Immediately following addition of test compounds, 10ml alamar blue (Thermo Fisher cat #DAL1100) was added to three of the medium only control wells on each plate. Cells were incubated for 2 hours at 37° C., 5% CO₂. Following the two-hour incubation, 110 ml sample from each of the control wells was removed and placed in a black, opaque 96-well plate. This plate was centrifuged for 5 minutes at 2000 RPM to remove any bubbles. Fluorescence was then measured (excitation=535nm, emission=595 nm) on a Molecular Devices FilterMax F5 microplate reader. Measured control fluorescence values (Cₛₜₐᵣₜ) serve as the baseline to measure assay endpoint proliferation. Plates were returned to 37° C., 5% CO₂ for 7 days (168 hours). Following incubation 10ml alamar blue was added to each test well as well as the other three control (medium only) wells. Following 2 hours incubation at 37° C., 5%CO₂, fluorescence was measured as described above. Endpoint control fluorescence values (C_{end}) and test well fluorescence values (Tend) were used to calculate the % of control proliferation using the following formula:

$$\% of control proliferation = ( ( {T}_{e n d} - {C}_{s t a r t} ) / ( {C}_{e n d} - {C}_{s t a r t} ) * 100$$

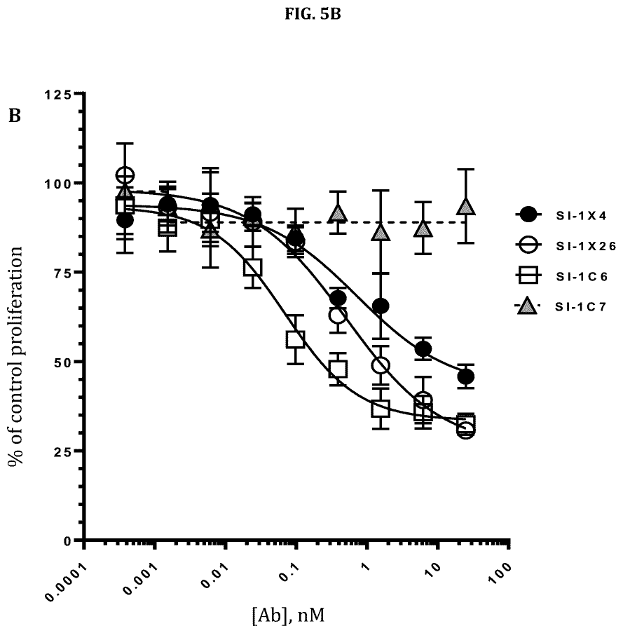

Data points were analysed by GraphPad Prism and inhibition curves were fitted by nonlinear regression [log(inhibitor) vs. response, 4 parameters] and IC₅₀ values were calculated. Data for cetuximab-based proteins is shown in FIG. 5A, while data for nimotuzumab-based proteins is shown in FIG. 5B. Fitted parameters for both sets of molecules are shown in Table 4. All the cetuximab-based bispecific antibodies (SI-1X6, SI-1X22, SI-1X24, SI-1X25) had similar inhibition of FaDu proliferation, which was more efficacious (64-76%) than that of the cetuximab control antibody (SI-1C6, 60%) and the anti-HER3 control Fc-scFv (SI-1C7, 9%). The nimotuzumab-based bispecific antibodies (SI-1X4, SI-1X26) had less potent inhibition of proliferation, consistent with the lower affinity of nimotuzumab for EGFR. Unexpectedly, the maximal inhibition of SI-1X26 was significantly higher than that of SI-1X4, suggesting that the geometry of SI-1X26 allows for more efficient blockade of EGFR and/or HER3 signalling compared to that of SI-1X4.

### Tables

Table 1 shows the characterization of EGFR x HER3 bispecific antibodies after a protein-A purification, including titer, purity (% protein of interest, POI), and thermal stability (melting temperature, Tm).

| | Titer | | |
|---|---|---|---|
| Protein | (ug/ml) | % POI | Tm (° C.) |
| SI-1X4 | 36.1 | 95.31 | 61.50 |
| SI-1X6 | 84.0 | 84.38 | 63.23 |
| SI-1X22 | 50.9 | 88.80 | 62.10 |
| SI-1X24 | 161.4 | 92.86 | 64.75 |
| SI-1X25 | 62.2 | 89.14 | 62.78 |
| SI-1X26 | 30.1 | 83.91 | 61.63 |

Table 2 shows EGFR binding kinetics of EGFR×HER3 bispecific antibodies as measured by the values of KD, kon, and kdis using biolayer interferometry.

| Protein | Response | KD (M) | kon(1/Ms) | kdis(1/s) |
|---|---|---|---|---|
| SI-1X4 | 0.1707 | 2.38E−08 | 7.16E+04 | 1.71E−03 |
| SI-1X6 | 0.4786 | 4.46E−09 | 3.12E+05 | 1.39E−03 |
| SI-1X22 | 0.4786 | 5.46E−09 | 2.94E+05 | 1.61E−03 |
| SI-1X24 | 0.4629 | 4.67E−09 | 2.52E+05 | 1.18E−03 |
| SI-1X25 | 0.4634 | 3.14E−09 | 2.45E+05 | 7.70E−04 |
| SI-1X26 | 0.1939 | 1.14E−08 | 5.51E+04 | 6.27E−04 |

Table 3 shows HER3 binding kinetics of EGFR×HER3 bispecific antibodies as measured by the values of KD, kon, and kdis using biolayer interferometry.

| Protein | Response | KD (M) | kon(1/Ms) | kdis(1/s) |
|---|---|---|---|---|
| SI-1X4 | 0.2643 | 1.56E−07 | 1.80E+05 | 2.81E−02 |
| SI-1X6 | 0.3068 | 1.22E−07 | 2.41E+05 | 2.92E−02 |
| SI-1X22 | 0.3090 | 1.64E−07 | 2.03E+05 | 3.32E−02 |
| SI-1X24 | 0.3718 | 1.06E−07 | 3.03E+05 | 3.21E−02 |
| SI-1X25 | 0.3807 | 9.38E−08 | 3.07E+05 | 2.88E−02 |
| SI-1X26 | 0.2941 | 1.51E−07 | 2.04E+05 | 3.08E−02 |

Table 4 shows the potency and efficacy parameters for EGFR×HER3 bispecific antibody-mediated inhibition of Fadu cell proliferation.

| Protein | IC50 (pM) | % Efficacy |
|---|---|---|
| SI-1X4 | 726.1 | 51.33 |
| SI-1X6 | 44.6 | 73.66 |
| SI-1X22 | 58.1 | 64.46 |
| SI-1X24 | 56.1 | 71.37 |
| SI-1X25 | 32.8 | 75.62 |
| SI-1X26 | 508.8 | 72.42 |
| SI-1C6 | 69.4 | 59.95 |
| SI-1C7 | 1.5 | 8.65 |

### Reference

1. Diaz-Serrano, A. et al. Genomic Profiling of HER2-Positive Gastric Cancer: PI3K/Akt/mTOR Pathway as Predictor of Outcomes in HER2-Positive Advanced Gastric Cancer Treated with Trastuzumab. Oncologist.23, 1092-1102 (2018).
2. Durkee, BY, et al. Cost-Effectiveness of Pertuzumab in Human Epidermal Growth Factor Receptor 2-Positive Metastatic Breast Cancer. Journal of Clinical Oncology. 2016, 34 (9): 902-9.
3. Gijsen, M. et al. HER2 Phosphorylation is Maintained by a PKB Negative Feedback Loop in Response to anti-HER2 Herceptin in Breast Cancer. PLOS Biol.8, e1000563 (2010).
4. Goel, S. & Winer, E. P. POINT: HER2-Targeted Combinations in Advanced HER2-Positive Breast Cancer. Oncology (Williston Park). 29, 797-798, 802 (2015).
5. Luque-Cabal, M. et al. Mechanisms Behind the Resistance to Trastuzumab in HER2-Amplified Breast Cancer and Strategies to Overcome It. Clin. Med. Insights Oncol. 10, 21-30 (2016).
6. McDonagh, C. F. et al. Antitumor Activity of a Novel Bispecific Antibody that Targets the ErbB2/ErbB3 Oncogenic Unit and Inhibits Heregulin-Induced Activation of ErbB3. Mol. Cancer Ther.11, 582-593 (2012).
7. R M Neve 1, U B Nielsen, D B Kirpotin, M A Poul, J D Marks, C C Benz. Biological effects of anti-ErbB2 single chain antibodies selected for internalizing function Biochem Biophys Res Commun. 2001, 280(1):274-9.
8. M K Robinson 1, K M Hodge, E Horak, A L Sundberg, M Russeva, C C Shaller, M von Mehren, I Shchaveleva, H H Simmons, J D Marks, G P Adams. Targeting ErbB2 and ErbB3 with a bispecific single-chain Fv enhances targeting selectivity and induces a therapeutic effect in vitro Br J Cancer 2008 Nov. 4;99(9): 1415-25.
9. Wang, Q. et al. The anti-HER3 Antibody in Combination with Trastuzumab Exerts Synergistic Antitumor Activity in HER2-positive Gastric Cancer. Cancer Lett.380, 20-30 (2016).
10. Yang, L. et al. NRG1-dependent Activation of HER3 Induces Primary Resistance to Trastuzumab in HER2-overexpressing Breast Cancer Cells. Int. J. Oncol.51, 1553-1562 (2017).
11. Cetuximab: https://www.ema.europa.eu/en/documents/scientific-discussion/erbitux-epar-scientific-discussion_en.pdf
12. Panitumumab: https://www.ncbi.nlm.nih.gov/pmc/articles/PMC6763619/#:˜: text=Pa nitumumab % 20binds % 20EGFR % 20with % 20an,whether % 20this % 20characteristic % 2 Ois % 20favorable
13. Nimotuzumab:https://www.nature.com/articles/s41598-019-57279-w/tables/1
14. Trastuzumab: https://www.ncbi.nlm.nih.gov/pmc/articles/PMC6244757/15.
15. Pertuzumab:https://www.tga.gov.au/sites/default/files/auspar-pertuzumab-131001.pdf
16. Patritumab: https://www.ncbi.nlm.nih.gov/pmc/articles/PMC5058629/17.
17. MM-121: https://www.ncbi.nlm.nih.gov/pmc/articles/PMC3478453/18.
18. MM-111:https://pubmed.ncbi.nlm.nih.gov/22248472/19.
19. 2in1: https://ars.els-cdn.com/content/image/1-s2.0-S1535610811003515-mmc1.pdf
20. SI-1X6.3(C3): US15/119,694.

[symbol]

What is claimed is:

1. A bispecific antibody, comprising two sets of heavy and light chains,
wherein each set of the heavy chain and the light chain form a Fab region having a binding specificity to EGFR,
wherein the antibody further comprises a scFv domain covalently linked to each set of the heavy chain and the light chain at N-terminal of the heavy chain, N-terminal of the light chain, or C-terminal of the light chain, and
wherein the scFv domain has a binding specificity to HER3.

2. The bispecific antibody of claim 1, wherein the scFv domain is linked to the N-terminal of the heavy chain, and wherein the antibody comprises an amino acid sequence having a sequence identity to SEQ ID NO. 22.

3. The bispecific antibody of claim 1, wherein the scFv domain is linked to the N-terminal or C-terminal of the light chain, and wherein the antibody comprises an amino acid sequence having a sequence identity to SEQ ID NO 17, 23, or 24.

4. The bispecific antibody of claim 1, comprising an antigen-binding domain having at least 98% sequence identity to SEQ ID NO. 17, 22, 23, or 24.

5. The bispecific antibody of claim 1, wherein the heavy chain comprises a constant region, wherein the constant region comprises an amino acid sequence having at least 98% sequence identity to SEQ ID NO. 19.

6. The bispecific antibody of claim 1, wherein the light chain comprises a kappa constant region, wherein the kappa constant region comprises an amino acid sequence having at least 98% sequence identity to SEQ ID NO. 20.

7. The bispecific antibody of claim 1, wherein the scFv domain, comprising an amino acid sequence having at least 98% sequence identity to SEQ ID NO. 11, 12, 13, 14, 15, or 16.

8. The bispecific antibody of claim 1, wherein the scFv domain comprises a variable light chain, wherein the variable light chain has an amino acid sequence having at least 98% sequence identity to SEQ ID NO. 11, 13, or 15.

9. The bispecific antibody of claim 1, wherein the scFv domain comprises a variable heavy chain, wherein the variable heavy chain has an amino acid sequence at least 98% sequence identity to SEQ ID NO. 12, 14, or 16.

10. The bispecific antibody of claim 1, wherein the scFv domain comprises a variable light chain (VL) and a variable heavy chain (VH), wherein the scFv domain has a configuration of V_{L}V_{H} or V_{H}V_{L} from the N terminal to the C terminal.

11. The bispecific antibody of claim 10, wherein the scFv domain comprises a disulphide bond between VL and VH.

12. The bispecific antibody of claim 11, wherein the disulfide bond is between vL100 and vH44 (Kabat) of the scFv domain.

13. The bispecific antibody of claim 1, wherein the scFv domain comprises R19S (Kabat) mutation.

14. The bispecific antibody of claim 1, wherein the antibody comprises an amino acid sequence having at least 98%sequence identity to SEQ ID NO. 17 and 18.

15. The bispecific antibody of claim 1, wherein the antibody comprises an amino acid sequence having at least 98%sequence identity to SEQ ID NO. 21 and 22.

16. The bispecific antibody of claim 1, wherein the antibody comprises an amino acid sequence having at least 98%sequence identity to SEQ ID NO. 18 and 23.

17. The bispecific antibody of claim 1, wherein the antibody comprises an amino acid sequence having at least 98%sequence identity to SEQ ID NO. 24 and 25.

18. An isolated nucleic acid encoding the bispecific antibody of claim 1.

19. An expression vector comprising the isolated nucleic acid of claim 18.

20. The expression vector of claim 19, wherein the vector is expressible in a cell.

21. cell comprising the nucleic acid of claim 18.

22. A method of producing the bispecific antibody of claim 1, comprising culturing the host cell of one of claim 21 so that the bispecific antibody is produced.

23. An immunoconjugate comprising the bispecific antibody of claim 1 and a cytotoxic agent, and wherein the cytotoxic agent comprises a chemotherapeutic agent, a growth inhibitory agent, a toxin, or a radioactive isotope.

24. A pharmaceutical composition, comprising the bispecific antibody of claim 1 and a pharmaceutically acceptable carrier.

25. The pharmaceutical composition of claim 24, further comprising radioisotope, radionuclide, a toxin, a therapeutic agent, a chemotherapeutic agent, or a combination thereof.

26. A pharmaceutical composition, comprising the immunoconjugate of claim 23 and a pharmaceutically acceptable carrier.

27. A method of treating a subject with a cancer, comprising administering to the subject an effective amount of the bispecific antibody of claim 1

28. The method of claim 27, wherein the cancer comprises cells expressing EGFR, HER3 or both, or wherein the cancer comprises breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukaemia, myeloma.

29. The method of claim 27, further comprising co-administering an effective amount of a therapeutic agent.

30. The method of claim 29, wherein the therapeutic agent comprises an antibody, a chemotherapy agent, an enzyme, or a combination thereof, and wherein the therapeutic agent comprises capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

31. The method of claim 27, wherein the subject is a human.

32. A solution comprising an effective concentration of the bispecific antibody of claim 1, wherein the solution is blood plasma in a subject.


SEQUENCE LISTING (96 sequences)

SEQ ID NO: 1 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 2 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 3 - protein, 107 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 4 - protein, 119 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSS

SEQ ID NO: 5 - protein, 107 aa, synthetic construct [sequence omitted]

SEQ ID NO: 6 - protein, 119 aa, synthetic construct [sequence omitted]

SEQ ID NO: 7 - protein, 112 aa, synthetic construct [sequence omitted]

SEQ ID NO: 8 - protein, 123 aa, synthetic construct [sequence omitted]

SEQ ID NO: 9 - protein, 107 aa, synthetic construct [sequence omitted]

SEQ ID NO: 10 - protein, 121 aa, synthetic construct [sequence omitted]

SEQ ID NO: 11 - protein, 111 aa, synthetic construct
QSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 12 - protein, 118 aa, synthetic construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 13 - protein, 113 aa, synthetic construct [sequence omitted]

SEQ ID NO: 14 - protein, 117 aa, synthetic construct [sequence omitted]

SEQ ID NO: 15 - protein, 111 aa, synthetic construct [sequence omitted]

SEQ ID NO: 16 - protein, 119 aa, synthetic construct [sequence omitted]

SEQ ID NO: 17 - protein, 473 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 18 - protein, 448 aa, synthetic construct
QVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 107 aa, synthetic construct [sequence omitted]

SEQ ID NO: 21 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 702 aa, synthetic construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 468 aa, synthetic construct
QVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 478 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGCGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 25 - protein, 452 aa, synthetic construct
QVQLQQSGAEVKKPGSSVKVSCKASGYTFTNYYIYWVRQAPGQCLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 219 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 706 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 28 - protein, 702 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 29 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 485 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 31 - protein, 5 aa, synthetic construct
NYGVH

SEQ ID NO: 32 - protein, 16 aa, synthetic construct
VIWSGGNTDYNTPFTS

SEQ ID NO: 33 - protein, 11 aa, synthetic construct
ALTYYDYEFAY

SEQ ID NO: 34 - protein, 11 aa, synthetic construct
RASQSIGTNIH

SEQ ID NO: 35 - protein, 7 aa, synthetic construct
YASESIS

SEQ ID NO: 36 - protein, 9 aa, synthetic construct
QQNNNWPTT

SEQ ID NO: 37 - protein, 5 aa, synthetic construct
NYYIY

SEQ ID NO: 38 - protein, 17 aa, synthetic construct
GINPTSGGSNFNEKFKT

SEQ ID NO: 39 - protein, 14 aa, synthetic construct
QGLWFDSDGRGFDF

SEQ ID NO: 40 - protein, 16 aa, synthetic construct
RSSQNIVHSNGNTYLD

SEQ ID NO: 41 - protein, 7 aa, synthetic construct
KVSNRFS

SEQ ID NO: 42 - protein, 9 aa, synthetic construct
FQYSHVPWT

SEQ ID NO: 43 - protein, 5 aa, synthetic construct
SYWMS

SEQ ID NO: 44 - protein, 17 aa, synthetic construct
NINRDGSASYYVDSVKG

SEQ ID NO: 45 - protein, 9 aa, synthetic construct
DRGVGYFDL

SEQ ID NO: 46 - protein, 14 aa, synthetic construct
TGTSSDVGGYNFVS

SEQ ID NO: 47 - protein, 7 aa, synthetic construct
DVSDRPS

SEQ ID NO: 48 - protein, 11 aa, synthetic construct
SSYGSSSTHVI

SEQ ID NO: 49 - DNA, 321 bp, synthetic construct
  source 1..321: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 50 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagc

SEQ ID NO: 51 - DNA, 321 bp, synthetic construct
  source 1..321: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 52 - DNA, 357 bp, synthetic construct
  source 1..357: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttct

SEQ ID NO: 53 - DNA, 321 bp, synthetic construct
  source 1..321: mol_type = other DNA
gacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggaaattaaa

SEQ ID NO: 54 - DNA, 357 bp, synthetic construct
  source 1..357: mol_type = other DNA
caggtgcagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagc

SEQ ID NO: 55 - DNA, 336 bp, synthetic construct
  source 1..336: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattact

SEQ ID NO: 56 - DNA, 369 bp, synthetic construct
  source 1..369: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagc

SEQ ID NO: 57 - DNA, 321 bp, synthetic construct
  source 1..321: mol_type = other DNA
gagatcgtgatgacccagagccccgccaccctgagcctgagccccggcgagcgggccaccctgagctgccgggccagccagagcgtgagcagctacctggcctggtaccagcagaagcccggccaggccccccggctgctgatctacgacgccagcaaccgggccaccggcatccccgcccggttcagcggcagcggcagcggcaccgacttcaccctgaccatcagcagcctggagcccgaggacttcgccgtgtactactgccaccagtacggcagcacccccctgaccttcggcggcggcaccaaggccgagatcaag

SEQ ID NO: 58 - DNA, 363 bp, synthetic construct
  source 1..363: mol_type = other DNA
caggtgcagctgcaggagagcggccccggcctggtgaagcccagccagaccctgagcctgacctgcaccgtgagcggcggcagcatcagcagcggcgactactactggagctggattcggcagccccccggcaagggcctggagtggatcggctacatctactacagcggcagcaccgactacaaccccagcctgaagagccgggtgaccatgagcgtggacaccagcaagaaccagttcagcctgaaggtgaacagcgtgaccgccgccgacaccgccgtgtactactgcgcccgggtgagcatcttcggcgtgggcaccttcgactactggggccagggcaccctggtgaccgtgagcagc

SEQ ID NO: 59 - DNA, 333 bp, synthetic construct
  source 1..333: mol_type = other DNA
cagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 60 - DNA, 354 bp, synthetic construct
  source 1..354: mol_type = other DNA
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagc

SEQ ID NO: 61 - DNA, 339 bp, synthetic construct
  source 1..339: mol_type = other DNA
gacatcgagatgacccagtctccagactccctggctgtgtctctgggcgagagggccaccatcaactgcaggtccagccagagtgttttatacagttccagcaatagaaactacttagcttggtaccagcagaatccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcgcacattcggacaagggaccaaagtggagatcaag

SEQ ID NO: 62 - DNA, 351 bp, synthetic construct
  source 1..351: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcggtagagacgtccaagaaccagttctccctgaagctgagctctgtgaccgccgcggacacggctgtgtattactgtgcgagagataaatggacttggtattttgacttatggggcagagggacactggtcaccgtctcttca

SEQ ID NO: 63 - DNA, 333 bp, synthetic construct
  source 1..333: mol_type = other DNA
caaagcgctctgactcaacccgcatctgtttcaggctctccagggcaatctatcacaatctcctgtaccggcacctctagcgacgtcggaagctacaacgttgtctcttggtatcaacagcacccaggaaaagcacccaagctgataatttacgaggtatcccagcgtcccagcggagtgagcaacagattttcaggttccaaatcaggtaatacagcaagtctgaccatctccggtcttcagactgaggacgaggctgactactattgctgttcctacgccggcagctctattttcgtcatttttggtggcgggacaaaagtgaccgtgctg

SEQ ID NO: 64 - DNA, 357 bp, synthetic construct
  source 1..357: mol_type = other DNA
gaagtgcagttgcttgagagtggaggcggacttgtccagcccggtgggtcactgcggctgtcttgcgctgcctccggttttaccttcagtcactatgtgatggcatgggtgcggcaggcccctggtaagggcctggagtgggtctcttccatttctagttcaggtgggtggaccttgtacgccgacagtgtgaagggacggttcactatctcacgggacaactcaaagaacacactctacttgcaaatgaatagtctcagggccgaggatacagccgtgtattactgcacacgcggtctgaagatggctacaatcttcgactactggggtcaggggactctggtgacagtcagctct

SEQ ID NO: 65 - DNA, 1422 bp, synthetic construct
  source 1..1422: mol_type = other DNA
gacatcctgctgacccagtctccagtgatcctgtccgtgtctcctggcgagagagtgtccttcagctgcagagcctctcagtccatcggcaccaacatccactggtatcagcagcggaccaacggctcccctcggctgctgattaagtacgcctccgagtctatcagcggcatcccctccagattctccggctctggctctggcaccgacttcaccctgtccatcaactccgtggaatccgaggatatcgccgactactactgccagcagaacaacaactggcccaccacctttggctgtggcaccaagctggaattgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccaggtgcagctgcaggagtcggggggaggcctggtcaagcctggagggtccctgagcctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtgtctagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 66 - DNA, 1347 bp, synthetic construct
  source 1..1347: mol_type = other DNA
caagttcagctcaagcagtctggccctggcctggttcagccttctcagagcctgagcatcacctgtaccgtgtccggcttctccctgaccaattacggcgtgcactgggttcgacagagccctggcaaatgcctggaatggctgggagtgatttggagcggcggcaacaccgactacaacacccctttcacctctcggctgtctatcaacaaggacaactccaagagccaggtgttcttcaagatgaactccctgcagtccaacgacaccgccatctactactgtgctcgggccctgacctactacgactacgagtttgcttactggggccagggcaccctggtcacagtttctgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 67 - DNA, 987 bp, synthetic construct
  source 1..987: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 68 - DNA, 321 bp, synthetic construct
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 69 - DNA, 645 bp, synthetic construct
  source 1..645: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 70 - DNA, 2109 bp, synthetic construct
  source 1..2109: mol_type = other DNA
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtgtctagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggcggtggagggtccggcggtggtggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtgtctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 71 - DNA, 1407 bp, synthetic construct
  source 1..1407: mol_type = other DNA
caggtgcaattgcaggagtcggggggaggcctggtcaagcctggagggtccctgagcctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggcggtggagggtccggcggtggtggatcagacatcctgctgacccagtctccagtgatcctgtccgtgtctcctggcgagagagtgtccttcagctgcagagcctctcagtccatcggcaccaacatccactggtatcagcagcggaccaacggctcccctcggctgctgattaagtacgcctccgagtctatcagcggcatcccctccagattctccggctctggctctggcaccgacttcaccctgtccatcaactccgtggaatccgaggatatcgccgactactactgccagcagaacaacaactggcccaccacctttggctgtggcaccaagctggaattgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 72 - DNA, 1437 bp, synthetic construct
  source 1..1437: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggttgtggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccaggtgcagctgcaggagtcggggggaggcctggtcaagcctggagggtccctgagcctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtgtctagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 73 - DNA, 1359 bp, synthetic construct
  source 1..1359: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagtgcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 74 - DNA, 660 bp, synthetic construct
  source 1..660: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 75 - DNA, 2121 bp, synthetic construct
  source 1..2121: mol_type = other DNA
caagttcaacttcaacaatctggtgctgaagttaaaaaacctggttcttctgttaaagtttcttgtaaagcctctggttatacttttactaattattatatttattgggttcgtcaagctcctggtcaaggtcttgaatggattggtggtattaatcctacttctggtggttctaattttaatgaaaaatttaaaactcgtgttactattactgccgatgaatcttccaccactgcttatatggaactttcttctcttcgttctgaagatactgctttttatttttgtacccgtcaaggtctttggtttgattctgatggtcgtggttttgatttttggggtcaaggtaccactgttactgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 76 - DNA, 2109 bp, synthetic construct
  source 1..2109: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 77 - DNA, 1347 bp, synthetic construct
  source 1..1347: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 78 - DNA, 1458 bp, synthetic construct
  source 1..1458: mol_type = other DNA
gagcccaaatcttccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 79 - DNA, 15 bp, synthetic construct
  source 1..15: mol_type = other DNA
aactatggtgtacac

SEQ ID NO: 80 - DNA, 48 bp, synthetic construct
  source 1..48: mol_type = other DNA
gtgatatggagtggtggaaacacagactataatacacctttcacatcc

SEQ ID NO: 81 - DNA, 33 bp, synthetic construct
  source 1..33: mol_type = other DNA
gccctcacctactatgattacgagtttgcttac

SEQ ID NO: 82 - DNA, 33 bp, synthetic construct
  source 1..33: mol_type = other DNA
agggccagtcagagtattggcacaaacatacac

SEQ ID NO: 83 - DNA, 21 bp, synthetic construct
  source 1..21: mol_type = other DNA
tatgcttctgagtctatctct

SEQ ID NO: 84 - DNA, 27 bp, synthetic construct
  source 1..27: mol_type = other DNA
caacaaaataataactggccaaccacg

SEQ ID NO: 85 - DNA, 15 bp, synthetic construct
  source 1..15: mol_type = other DNA
aactactacatctac

SEQ ID NO: 86 - DNA, 51 bp, synthetic construct
  source 1..51: mol_type = other DNA
ggcatcaaccccaccagcggcggcagcaacttcaacgagaagttcaagacc

SEQ ID NO: 87 - DNA, 42 bp, synthetic construct
  source 1..42: mol_type = other DNA
cagggcctgtggttcgacagcgacggccggggcttcgacttc

SEQ ID NO: 88 - DNA, 48 bp, synthetic construct
  source 1..48: mol_type = other DNA
cgttcttctcaaaatattgttcattctaatggtaatacttatcttgat

SEQ ID NO: 89 - DNA, 21 bp, synthetic construct
  source 1..21: mol_type = other DNA
aaagtttctaatcgtttttct

SEQ ID NO: 90 - DNA, 27 bp, synthetic construct
  source 1..27: mol_type = other DNA
tttcaatattctcatgttccttggact

SEQ ID NO: 91 - DNA, 15 bp, synthetic construct
  source 1..15: mol_type = other DNA
agttattggatgagc

SEQ ID NO: 92 - DNA, 51 bp, synthetic construct
  source 1..51: mol_type = other DNA
aacataaaccgcgatggaagtgcgagttactatgtggactctgtgaagggc

SEQ ID NO: 93 - DNA, 27 bp, synthetic construct
  source 1..27: mol_type = other DNA
gatcgtggggtgggctacttcgatctc

SEQ ID NO: 94 - DNA, 42 bp, synthetic construct
  source 1..42: mol_type = other DNA
actggaaccagcagtgacgttggtggttataactttgtctcc

SEQ ID NO: 95 - DNA, 21 bp, synthetic construct
  source 1..21: mol_type = other DNA
gatgtcagtgatcggccctca

SEQ ID NO: 96 - DNA, 33 bp, synthetic construct
  source 1..33: mol_type = other DNA
agctcatatgggagcagcagcactcatgtgatt